(19) United States
(12) Patent Application Publication
(10) Pub. No.: US 2023/0147401 A1
(43) Pub. Date: May 11, 2023

(54) METHOD TO PURIFY TRIS-(3-HYDROXYBUTYRATO)-GLYCERYL ESTER

(71) Applicant: DR. SCHAR S.P.A., Postal (IT)

(72) Inventors: Virna Lucia Cerne, Postal (IT); Gabriele Razzetti, Sesto San Giovanni (IT); Simone Mantegazza, Milano (IT); Roberto Rossi, Milano (IT); Philippe Carboni, Rimini (IT); Niccolo Santillo, Siena (IT); Davide Brenna, Senago (IT); Emanuele Attolino, Palagiano (IT)

(73) Assignee: DR. SCHAR S.P.A., Postal (IT)

(21) Appl. No.: 17/767,621

(22) PCT Filed: Oct. 6, 2020
(86) PCT No.: PCT/IT2020/050246

§ 371 Date: Apr. 8, 2022

(30) Foreign Application Priority Data
Oct. 10, 2019 (IT) 102019000018491

(51) Int. Cl. C07C 67/58; C07C 67/31

(52) U.S. Cl. CPC C07C 67/58; C07C 67/31

(57) ABSTRACT

The present invention concerns a method to prepare and purify the ester glyceryl-tris-(3-hydroxybutyrate) of formula (I): and its optically active isomers, in particular the enantiomer (R, R, R).

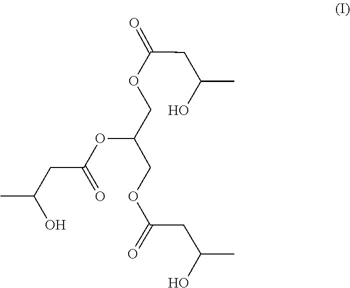 [SMILES: CC(O)CC(=O)OCC(COC(=O)CC(C)O)OC(=O)CC(C)O]

### FIELD OF THE INVENTION

The present invention concerns a method to prepare and purify glyceryl-tris-(3-hydroxybutyrate) ester, which is used in diets that require specific nutritional needs, such as for example the ketogenic diet.

### BACKGROUND OF THE INVENTION

Glyceryl-tris-(3-hydroxybutyrate) ester, having the following structural formula (I):

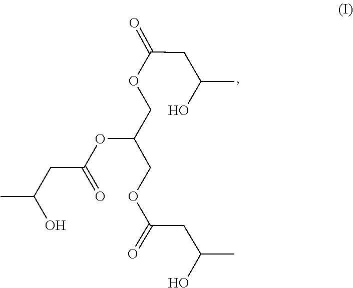 [SMILES: CC(O)CC(=O)OCC(COC(=O)CC(C)O)OC(=O)CC(C)O]

and in particular its enantiomer having all three stereogenic centers in configuration (R) of formula (Ia)

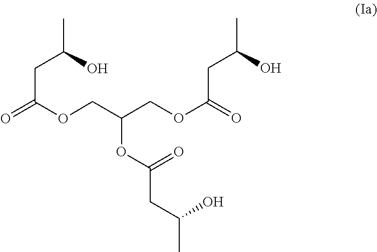 [SMILES: C[C@@H](O)CC(=O)OCC(COC(=O)C[C@@H](C)O)OC(=O)C[C@@H](C)O]

are used in the ketogenic diet (KD) which is a nutritional approach consisting of a high-fat content and low carbohydrate levels. A diet high in fat but low in carbohydrates leads to ketosis, where the body gains energy from fat in the form of ketone products. This type of diet has been used in the treatment of various diseases, for example in the treatment of infantile refractory epilepsy (E. van der Louw et al. Eur. J. Paediatr. Neurol. 2016 20, 798-809).

Patent application WO 95/09144 describes a method to obtain glyceryl-tris-(3-hydroxybutyrate) ester of formula (I) which provides a first esterification of the glycerin of fonnula (II) with tert-butyl acetoacetate of formula (III) and subsequent hydrogenation of the intermediate of formula (IV) in the presence of Raney nickel as catalyst.

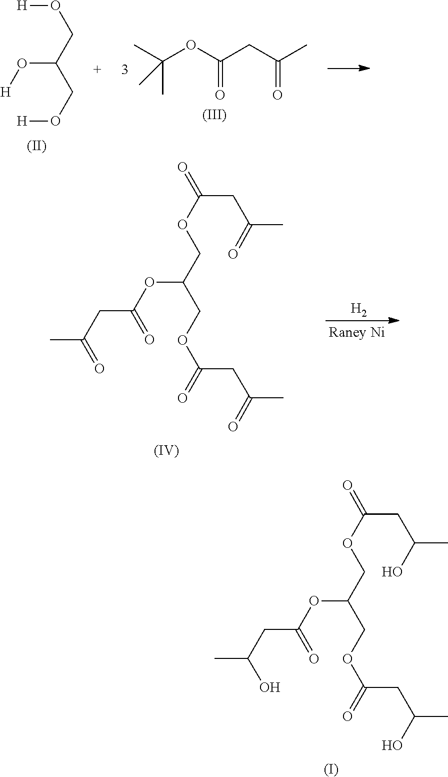

While the condensation of the glycerin of formula (II) and the acetoacetate of formula (III) continues with a good yield, the hydrogenation in the presence of Raney nickel as catalyst requires a pressure of 1000 psi, therefore about 70 bar, which is a very high pressure, especially in the perspective of production on an industrial scale. Furthermore, patent application WO 95/09144 describes the purification of the desired product by means of ion exchange resins necessary to remove the residual nickel in the product.

Patent application JP.03-083950 describes a method to obtain the optically active compound of formula (Ia) by means of a sequence of reactions which provide the initial protection of methyl 3-(R)-hydroxybutyrate of formula (V) as THP and subsequent hydrolysis to obtain the acid of formula (VI). The carboxylic acid is then activated by treatment with CDI and then esterified with the glycerin of formula (II) to obtain the protected intermediate of formula (VII). The deprotection reaction by the tetrahydropyranyl protective groups of the compound of formula (VII) in an acid environment finally leads to the optically active product of formula (Ia):

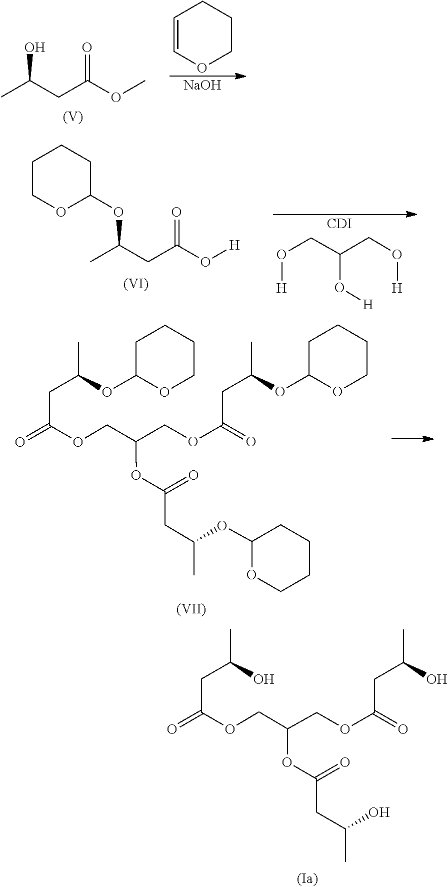

The method described in JP.03-083950 is a long method and leads to the desired product with low yields. Furthermore, the product of formula (Ia) is purified by chromatography to obtain the pure compound.

Both the compound of formula (I) and its optically active isomer (Ia) at room temperature present as oils, as in all cases known to the person of skill in the art for triglycerides of short-chain fatty acids. Since it is not possible to purify the compounds by crystallization, and since it is desired to avoid silica chromatography on an industrial scale, also the purification of a compound of formula (I) and formula (Ia) to obtain products having a suitable purity higher than 99% in A% HPLC is very complex, and so far it has not been reported in the literature that such a degree of purity has been achieved with different methods. There is therefore a need for an alternative, simpler and more advantageous method to prepare glyceryl-tris-(3-hydroxybutyrate) ester of formula (I) and its optically active isomer having all three stereogenic centers with a configuration (R) of formula (Ia). This new method should in particular provide a smaller number of synthetic steps, avoiding the extensive use of protective groups, and should improve the atom economy of the process. The method should also be economical, safe for humans and the environment, use mild reaction conditions and at the same time provide the desired compounds in high yields and high chemical and stereochemical purity.

### SUMMARY OF THE INVENTION

The invention concerns a method to prepare a compound of formula (I)

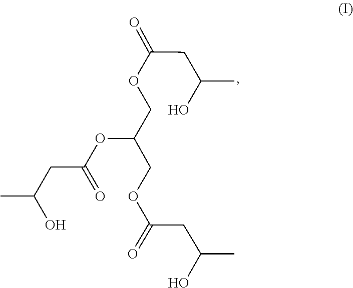 [SMILES: CC(O)CC(=O)OCC(COC(=O)CC(C)O)OC(=O)CC(C)O]

as a single enantiomer or as a mixture of isomers, comprising the hydrogenation reaction of a compound of formula (IV)

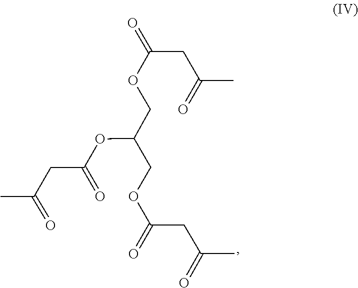 [SMILES: CC(=O)CC(=O)OCC(COC(=O)CC(C)=O)OC(=O)CC(C)=O]

in the presence of a ruthenium-based catalyst.

Another purpose of the present invention in a method to purify a compound of formula (I) as defined above comprising:
a. one or more washes of an aqueous solution of a compound of formula (I) and wherein the aqueous solution typically comprises NaCl from 0% to 5% w/w, with an organic solvent S1, as defined here;
b. increase of the concentration of NaCl of the aqueous solution of a compound of formula (I) as in point a. greater than 5% w/w;
c. one or more extractions of the aqueous solution of a compound of formula (I) as in point b. with s solvent S2, as defined here and
d. concentration of the solution of a compound of formula (I) in a solvent S2 to obtain a compound of formula (I) as transparent oil.

The organic solvent S1 is typically an organic solvent selected from a cyclic or acyclic ether or a non-polar aprotic solvent.

The organic solvent S2 is typically an organic solvent selected from a polar aprotic solvent; a chlorinated solvent; an ester; or a linear or branched C₃-C₇ ketone.

### DETAILED DESCRIPTION OF THE INVENTION

The invention concerns a method to prepare a compound of formula (I)

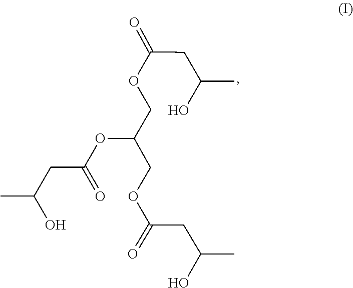 [SMILES: CC(O)CC(=O)OCC(COC(=O)CC(C)O)OC(=O)CC(C)O]

as a single enantiomer or as a mixture of isomers, comprising the hydrogenation reaction of a compound of formula (IV)

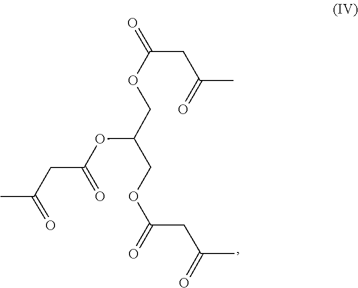 [SMILES: CC(=O)CC(=O)OCC(COC(=O)CC(C)=O)OC(=O)CC(C)=O]

in the presence of a ruthenium-based catalyst.

The hydrogenation of a compound of formula (IV) can be carried out by catalytic hydrogenation in the presence of a homogeneous or heterogeneous Ru-based metal catalyst.

When the metal catalyst is heterogeneous, it is preferably deposited on an inert support such as, for example, carbon, barium hydroxide, alumina, calcium carbonate; preferably carbon. The concentration of the metal on the support can vary between about 1 and 30%, preferably between about 5 and 20%.

The hydrogen pressure employed can vary between about 1 bar and about 50 bar, preferably between 2 bar and 40 bar, for example, at 3 bar, 4 bar, 5 bar, 6 bar, 7 bar, 8 bar, 9 bar, 10 bar, 15 bar, 20 bar, 25 bar, 30 bar or 35 bar.

The molar quantity of catalyst used, referred to the compound of formula (IV), is comprised between about 0.1 and 10%, preferably between about 0.5 and 5%.

The hydrogenation reaction can be carried out in the presence of an organic solvent selected, for example, from a polar aprotic solvent, typically dimethylformamide, dimethylacetamide, acetonitrile, dimethyl sulfoxide; a cyclic or acyclic ether, typically tetrahydrofuran or dioxane or methyl tert-butyl ether; a chlorinated solvent, typically dichloromethane; a non-polar aprotic solvent, typically toluene or hexane; a polar protic solvent, such as a linear or branched C₁-C₆ alcohol, in particular methanol, ethanol, isopropanol or butanol; an ester, for example ethyl acetate, isopropyl acetate, butyl acetate; a carboxylic acid, for example acetic acid or propionic acid; or water; or mixtures of two or more of said solvents, preferably 2 or 3.

Preferably, the reaction can be carried out in a C₁-C₆ alcohol, for example ethanol or isopropanol, in an ester solvent, for example ethyl acetate, or in a mixture of an ester solvent, for example ethyl acetate, and water.

This hydrogenation reaction can be carried out at a temperature comprised between about 0° C. and the reflux temperature of the solvent; preferably between about 25° C. and the reflux temperature.

The hydrogenation reaction of a compound of formula (IV) can also be carried out by means of a hydrogen transfer reaction, using a homogeneous or heterogeneous metal catalyst, for example as defined above, and in the same molar quantity, and a hydrogen donor. The latter selected for example in the group comprising cyclohexene; cyclohexadiene; methylcyclohexene; limonene; dipentene; mentene; hydrazine; phosphinic acid or its derivatives, for example sodium hypophosphite; indoline; ascorbic acid; formic acid or its sodium or ammonium salts; and secondary alcohols, for example isopropanol.

The molar ratio between the hydrogen donor and the compound of formula (IV) can be comprised between about 1.5 and 50, preferably between about 1.5 and 10.

The hydrogen transfer reduction reaction can be carried out in the presence of an organic solvent, selected for example from one of the solvents mentioned above.

In a preferred aspect of the invention the catalyst is heterogeneous and is more preferably Ru/C.

In another preferred aspect of the invention the catalyst is homogeneous and is more preferably a Ruthenium complex with mono or diphosphine ligands well known in the chemistry of enantioselective hydrogenations, for example the homogeneous catalyst Ru((R)-BINAP)Cl₂.

In accordance with a preferred aspect of the invention, the hydrogenation of a compound of formula (IV) using the homogeneous catalyst Ru((R)-BINAP)Cl₂ allows to obtain a compound of formula (Ia)

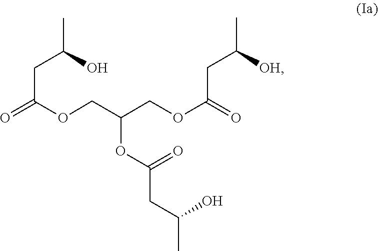 [SMILES: C[C@@H](O)CC(=O)OCC(COC(=O)C[C@@H](C)O)OC(=O)C[C@@H](C)O]

having all three stereogenic centers in configuration (R).

Therefore, according to another aspect, the invention provides an advantageous method to prepare a compound of formula (Ia)

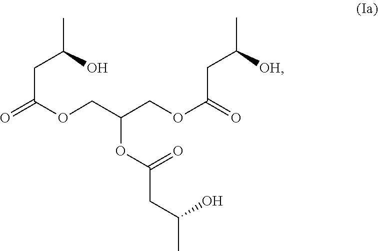 [SMILES: C[C@@H](O)CC(=O)OCC(COC(=O)C[C@@H](C)O)OC(=O)C[C@@H](C)O]

as defined here, comprising the hydrogenation reaction of a compound of formula (IV)

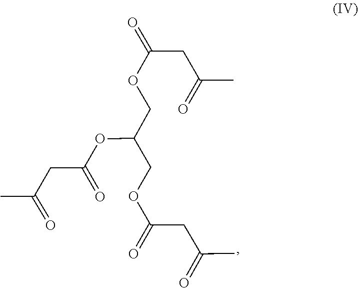 [SMILES: CC(=O)CC(=O)OCC(COC(=O)CC(C)=O)OC(=O)CC(C)=O]

in the presence of the homogeneous catalyst Ru((R)-BINAP)Cl₂.

A compound of formula (IV) is a known compound and can be obtained for example by esterification reaction of alvcerin of formula (II)

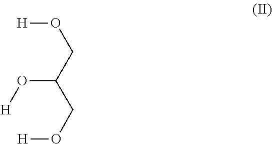

with tert-butyl acetoacetate of formula (III)

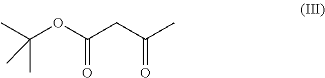

Compounds of formula (II) and formula (III) are commercially available.

At the end of the hydrogenation, both the glyceryl-tris(-3-hydroxybutyrate) ester of formula (I) as well as its enantiomer of formula (Ia) are obtained by evaporation of the solvent as liquids having a purity measured by means of HPLC always higher than 90%, but lower than 99%.

It has been surprisingly found that the glyceryl-tris(-3-hydroxybutyrate) ester of formula (I) and its enantiomer of formula (Ia), unlike common triglycerides which are lipophilic and insoluble in water such as the compound of formula (IV), have an amphiphilic nature which makes them soluble under specific experimental conditions both in water and also in its saline solutions as well as in organic solvent.

The present invention also concerns a method to purify a compound of formula (I) or of formula (Ia) comprising:
a. one or more washes of an aqueous solution of a compound of formula (I) or of formula (Ia) and wherein the aqueous solution comprises NaCl from 0% to 5% w/w, preferably from 1% to 5% w/w, with an organic solvent S1, as defined here;
b. increase of the concentration of NaCl of the aqueous solution of a compound of formula (I) or (Ia) as in point a. greater than 5% w/w, preferably at least 10% w/w, more preferably at least 15% w/w;
c. one or more extractions of the aqueous solution of a compound of formula (I) or of formula (Ia) as in point b. with an organic solvent S2, as defined here; and
d. concentration of the solution of a compound of formula (I) or formula (Ia) in an organic solvent S2 in order to obtain a compound of formula (I) or of formula (Ia) as a transparent oil, typically with a purity measured by means of HPLC greater than 99%.

A solvent S1 is an organic solvent selected, for example, from a cyclic or acyclic ether, typically diethyl ether or methyl tert-butyl ether, typically methyl tert-butyl ether; a non-polar aprotic solvent, typically toluene.

A solvent S2 is an organic solvent selected, for example, from a polar aprotic solvent, typically acetonitrile; a chlorinated solvent, typically dichloromethane; an ester, for example ethyl acetate, isopropyl acetate, butyl acetate, preferably ethyl acetate; a linear or branched C₃-C₇ ketone, for example, methyl ethyl ketone, methyl isobutyl ketone.

The washing with the solvent S1 or the extraction with the solvent S2 of a compound of formula (I) or of formula (Ia) can be carried out at a temperature comprised between about 0° C. and about 60° C.; preferably between about 25° C. and about 60° C., for example at 30° C., at 35° C., at 40° C., at 45° C., at 50° C. or at 55° C.

The solution of a compound of formula (I) or of formula (Ia) in a solvent S2 can be anhydrated by drying. Drying can be carried out by means of anhydration with a dehydrating agent, for example sodium sulfate (Na₂SO₄), magnesium sulfate (MgSO₄) or anhydrous calcium chloride (CaCl₂), preferably sodium sulfate (Na₂SO₄).

A compound of formula (I) or formula (Ia) as a transparent oil with a purity measured by means of HPLC greater than 99% has never been obtained without the aid of chromatographic purification techniques.

The present invention also concerns a method to purify a compound of formula (I) or of formula (Ia) with a purity measured by means of HPLC greater than 99% without the aid of purification techniques of the chromatographic type, for example without proceeding with chromatographic purification on ion exchange resin or normal or inverse stationary phases.

The extraction in an organic solvent of a compound of formula (I) or of formula (Ia) from an aqueous solution in accordance with point c. of the purification process also allowed to bring down the content of all heavy metals and of ruthenium in particular in a compound of formula (I) or formula (Ia) well below the limits provided by the ICH guidelines, and which previously had only been possible by means of chromatographic purification on ion exchange resin.

Therefore, the present invention also concerns a compound of formula (I) or formula (Ia), obtained in accordance with the process and purification method object of the present invention, having a content of heavy metals lower than 0.5 ppm.

The following examples further illustrate the invention:

### EXAMPLE 1

### Synthesis of Glycerol Tris-Acetoacetate of Formula (IV)

Glycerin (150 g, 1.69 mol), tert-butyl acetoacetate (1350 g, 8.55 mol) are added to a 3000 ml flask in inert atmosphere and the mixture is heated at 95-100° C. for 2.5 hours. The reaction mixture is then concentrated at a reduced pressure of 200-250 mbar and at an internal temperature of 80-90° C., integrating toluene in portions to the reaction mixture for a total of 1.5 1. The end of distillation residue is then cooled at 0-10° C. and diluted with isopropanol cooled at −10° C. (2.5 1), the phases are separated, discarding the alcoholic phase and obtaining a crude oil (620 g) which is repeatedly washed with cold isopropanol until a product (400 g) is obtained with an HPLC purity (200 nm) in A% of 98.6% with a glycerol bis-acetoacetate content lower than 0.3% and a yield of 70%.

¹-NMR (CDCl₃, 300 MHz) δ: 11.8*(s, 1H); 5.34 (m, 1H); 4.99*(s, 1H); 4.32 (m, 4H); 3.49 (s, 6H); 2.25 (s, 9H); 1.96*(s, 3H). *Keto-enol isomerism.

HPLC-MS: 345 (M/z+1)

### EXAMPLE 2

### Synthesis of Glyceryl-Tris(-3-hydroxybutyrate) Ester of Formula (I)

A solution of glycerol tris-acetoacetate of formula (IV) (100 g, 0.29 mol) in ethyl acetate (500 ml) and the ruthenium on carbon at 5% with water content approximately 50% (30 g) are loaded into a 1000 ml autoclave at room temperature. The autoclave is inerted with nitrogen and after vacuuming it, it is pressurized with hydrogen at 4.5-5 bar and 1000 rpm of stirring for 6-8 hours. The reaction is monitored by means of HPLC analysis and when the reaction is complete the reactor is inerted and the catalyst is filtered on perlite, washing with ethyl acetate (100 ml). The solution is concentrated to residue at reduced pressure and at a temperature of 30-35° C. The crude is dissolved in water (350 ml), treated with decoloring carbon (2.5 g), left under stirring for 2 hours then filtered on perlite and washed with water (150 ml). Sodium chloride (25 g) and methyl tert-butyl ether (140 ml) are added to the aqueous phase. The two phases are left under vigorous stirring for 30 minutes at a temperature of 45-50° C. and are separated, discarding the organic phase. More sodium chloride (50 g) and ethyl acetate (400 ml) are added to the aqueous phase. The phases are left under stirring at 45-50° C. for 30 minutes and the phases are separated. The aqueous phase is extracted with further ethyl acetate and the organic phases are reunited, anhydrated on sodium sulfate and concentrated to residue at a reduced pressure at a temperature of 30-35° C. The washing and extraction procedure is repeated 3 times obtaining 72 g of the compound of formula (I) as a colorless oil with HPLC purity (200 nm) at 99.3% in A% and a yield of 71%.

¹H-NMR (CDCl₃, 300 MHz) δ: 5.32 (m, 1H); 4.44-4.10 (m, 7H); 2.76 (s, 3H); 2.52-2.35 (m, 6H); 1.21 (d, 9H).

HPLC-MS: 351 (M/z+1)

### EXAMPLE 3

### Synthesis of Glyceryl-Tris-(3- R)-Hydroxybutyrate) Ester of Formula (Ia)

A solution of glycerol tris-acetoacetate (150 g, 0.43 mol) and the catalyst Ru((R)-BINAP)Cl₂ (0.69 g, 0.87 mmol) in ethanol (500 ml) are loaded into a 1000 ml autoclave at room temperature. The autoclave is inerted with nitrogen, heated at 40-45° C. and after vacuuming it is pressurized with hydrogen at 35 bar and 1600 rpm for 6-8 hours. The disappearance of the starting product is verified by means of HPLC analysis, the autoclave is unloaded, and the solution is filtered on a perlite and carbon panel. The solution filtered to residue is concentrated at a temperature of 45-50° C. and at a reduced pressure. The product is dissolved in water (500 ml) and decoloring carbon (3.75 g) is added, leaving it under vigorous stirring for 2 hours at room temperature. The crude solution is filtered on a perlite panel washing with water (225 ml) and the aqueous solution is used in the purification step.

¹H-NMR (CDCl₃, 300 MHz) δ: 5.32 (m, 1H); 4.44-4.10 (m, 7H); 2.76 (s, 3H); 2.52-2.35 (m, 6H); 1.21 (d, 9H).

HPLC-MS: 351 (M/z+1).

### EXAMPLE 4

### Purification of Glyceryl-Tris-(3-(R)-Hydroxybutyrate) Ester of Formula (Ia)

Sodium chloride (36 g) is added to the solution obtained in Example 3, it is heated at the temperature of 45-50° C. and the aqueous phase is washed with toluene (2×225 ml) and subsequently with methyl tert-butyl ether (2×200 ml). Sodium chloride (180 g) is added to the aqueous solution and the product is extracted with ethyl acetate (2×200 ml), the reunited organic phases are anhydrated with sodium sulphate, filtered on perlite and concentrated to residue at the temperature of 45-50° C. and at reduced pressure. 120 g of glyceryl-tris-(3-(R)-hydroxybutyrate) ester of formula (Ia) are obtained, with an HPLC purity (200 nm) of 99.1% in A% and a yield of 79%. Rotary optical power

[symbol]

### (C=1.15 Methanol)=−22.0°.

1. A method of purifying a compound of formula (I) 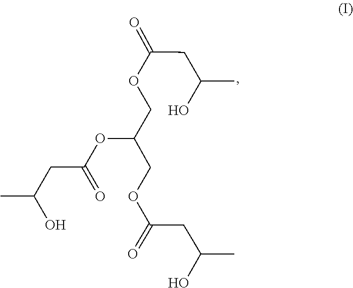 [SMILES: CC(O)CC(=O)OCC(COC(=O)CC(C)O)OC(=O)CC(C)O]
as a single enantiomer or as a mixture of isomers, comprising:
a. one or more washes of an aqueous solution of a compound of formula (I) and wherein the aqueous solution comprises NaCl from 0% to 5% w/w, with an organic solvent S1;
b. increase of the concentration of NaCl of the aqueous solution of a compound of formula (I) as in point a. greater than 5% w/w;
c. one or more extractions of the aqueous solution of a compound of formula (I) as in point b. with an organic solvent S2, and
d. concentration of the solution of a compound of formula (I) in an organic solvent S2 to obtain a compound of formula (I),
and wherein the solvent S1 is an organic solvent selected from a cyclic or acyclic ether or a non-polar aprotic solvent, and wherein the solvent S2 is an organic solvent selected from a polar aprotic solvent; a chlorinated solvent; an ester; or a linear or branched C₃-C₇ ketone.

2. The method of claim 1, wherein the compound of formula (I) is a compound of formula (Ia),

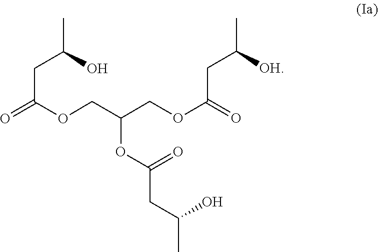 [SMILES: C[C@@H](O)CC(=O)OCC(COC(=O)C[C@@H](C)O)OC(=O)C[C@@H](C)O]

3. The method of claim 1, wherein the aqueous solution as in point a. comprises NaCl from 1% to 5% w/w.

4. The method of claim 1, wherein the aqueous solution as in point b. comprises NaCl at least 10% w/w.

5. The method of claim 4, wherein the aqueous solution as in point b. comprises NaCl at least 15% w/w.

6. The method claim 1, wherein the organic solvent S1 is diethyl ether, methyl tert-butyl ether, or toluene.

7. The method of claim 1, wherein the organic solvent S2 is acetonitrile, dichloromethane, ethyl acetate, isopropyl acetate, butyl acetate, methyl ethyl ketone, or methyl isobutyl ketone.

8. The method claim 1, wherein none of the steps comprises a purification by means of chromatography.

9. The method claim 1, wherein the compound of formula (I) is obtained by means of a method comprising the hydrogenation reaction of a compound of formula (IV) 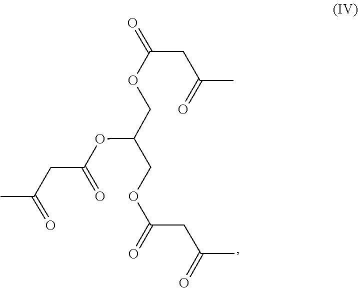 [SMILES: CC(=O)CC(=O)OCC(COC(=O)CC(C)=O)OC(=O)CC(C)=O]
in the presence of a ruthenium-based catalyst.

10. The method claims 2, wherein the compound of formula (I), as defined in claim 1, or the compound of formula (Ia), as defined in claim 2, have a content of heavy metals less than 0.5 ppm.
